(19) United States
(12) Patent Application Publication
(10) Pub. No.: US 2025/0064681 A1
(43) Pub. Date: Feb. 27, 2025

(54) MEDICATION TRACKING SYSTEM AND APPARATUS

(71) Applicant: Arunasri Nishtala, Poolesville, MD (US)

(72) Inventor: Arunasri Nishtala, Poolesville, MD (US)

(21) Appl. No.: 18/813,932

(22) Filed: Aug. 23, 2024

Related U.S. Application Data
(60) Provisional application No. 63/578,168, filed on Aug. 23, 2023.

(51) Int. Cl. A61J 7/04; A61J 1/03; A61J 1/14; A61J 7/02; G16H 20/10

(52) U.S. Cl. CPC A61J 7/0436; A61J 1/035; A61J 1/1412; A61J 7/02; A61J 7/0481; G16H 20/10; A61J 2200/30; A61J 2200/70; A61J 2200/74; A61J 2205/10

(57) ABSTRACT

A medication tracking system and apparatus, configured to facilitate the conscious self-administration of medicine by a user so as to prevent skipped doses, overdose, and missing/stolen medication. The system employs a mobile device application running on a mobile device of the user, which connects wirelessly to a tracking apparatus disposed within a lid of a medicine container or medicine vial. The system and apparatus are configured to enable patients to easily record their medication adherence without needing to rely of memory or counting pills. Scales within the vial and/or pill container detect when a dose is taken, and information is conveyed from the apparatus to the mobile device via Bluetooth™.

### CONTINUITY

This application is a non-provisional patent application of provisional patent application No. 63/578,168, filed on Aug. 23, 2023, and priority is claimed thereto.

### FIELD OF THE PRESENT INVENTION

The present invention relates to the field of medicine, and more specifically relates to a system and apparatus for tracking medication doses as prescribed by a physician which are self-administered by a user.

### BACKGROUND OF THE PRESENT INVENTION

Countless individuals all over the world are prescribed medication to the benefit of their health. Many medications are critical to sustaining a patient's life. Others are configured to mitigate pain. Such medications, taken as prescribed, are quite helpful.

Unfortunately, some individuals fail to take their medications as prescribed. Some people accidentally skip doses. Others are prone to forgetting that a dose was taken, and ultimately take a second dose which can have adverse consequences. Other patients may accidentally take the wrong medicine and be unaware of the mistake. Further, medication overdose is a huge concern, as it can lead to the death of the patient or other severe side effects. Additionally, monitoring cases of intentional overdose of painkillers is critical.

Often, medical practitioners and clinical researchers need data to provide insight into treatments for conditions and diseases. This data often pertains to the quantity and types of medications susceptible individuals have been prescribed over the course of their lives. For example, if one is studying Alzheimer's disease, they will want to know what medications were taken over their entire life to try to gain insight into what might be partial causes for the development of the disease. Unfortunately, obtaining accurate medication intake data, especially over the course of an entire lifetime is essentially impossible with current medication tracking systems. Additionally, even if a complete medical history of the individual is available, it generally does not confirm that the individual actually took the medication that was prescribed in the prescribed doses, and at the prescribed intervals. If there were a way to capture medication intake data for individuals in a reliable and secure fashion, medical research could be better informed.

While there are several healthcare digital mobile device applications available for patients, they do not have a seamless method of recording a patient's individual medication intake. Conventionally, patients must employ their phone to manually record their intake. This is subject to error as patients may forget to record the intake manually on the application, or the time at which they record is not always the exact time the intake occurred. Further, none of the products presently on the market are configured to weigh the remaining medication within a medicine container to determine if and when medication has been removed from the container, and none of the present products facilitate automatically informing a physician or pharmacist of the progression of the timely intake of the prescribed medication by the physician or pharmacist.

If there were an easy way in which medication dosages could be seamlessly tracked with minimal effort on behalf of the patient, accidents such as taking too much medicine, taking too little medicine, or taking the wrong medicine could be minimized. Further, if there were a way in which users could track when a medicine is dispensed from a medicine bottle, medicine vial, and/or medicine blister pack, medicine theft and inadvertent overdose could be mitigated.

Thus, there is a need for a new system by which medication dosages may be tracked by patients such that they are taken as prescribed. Such a system preferably employs a mobile device application, running on a mobile device of a patient, which is configured to connect to an apparatus integrated into the medicine container, medicine vial, and/or medicine blister pack which conveys medication intake to the mobile device application wirelessly, thereby facilitating the tracking of medication intake in real-time.

Additionally, there is a need for a new system configured to employ QR codes to seamlessly link prescription information for the medication of the medicine container to the patient on the mobile device application running on the mobile device of the patient.

### SUMMARY OF THE INVENTION

The present invention is a system and apparatus configured to track medication intake of a patient. The apparatus is equipped with sensors, including, but not limited to, a scale, a blister pack pill counter, and a motion detector, to provide insight to the system as to when the medicine bottle, medicine vial, and/or blister pack is accessed. The scale is configured to monitor the weight of the medication contained therein to determine if/when medication is dispensed. Such medication information is relayed to the mobile device and the data is stored to a database of the mobile device application. QR codes are preferably employed for initial setup such that the mobile device application is aware of what medication is prescribed and the prescribed dosage (amount, time of day, and frequency). The mobile device application displays to the patient, and in some cases, to the physician of the patient, the success or failure of the adherence to the medication dosage and schedule. Further, the mobile device application may be configured to inform the patient of potential side effects of the prescribed medication, and to warn of potential drug interactions.

The following brief and detailed descriptions of the drawings are provided to explain possible embodiments of the present invention but are not provided to limit the scope of the present invention as expressed herein this summary section.

### BRIEF DESCRIPTION OF DRAWINGS OF THE PRESENT INVENTION

The accompanying drawings, which are incorporated herein and form a part of the specification, illustrate the present invention and, together with the description, further serve to explain the principles of the invention and to enable a person skilled in the pertinent art to make and use the invention.

The present invention will be better understood with reference to the appended drawing sheets, wherein:

FIG. 1 details a view of a medication canister preferred embodiment of the present invention as seen from the side.

FIG. 2 depicts a view of a medication vial preferred embodiment of the present invention as seen from the side.

FIG. 3 exhibits a flow chart detailing the process of use of the system and apparatus of the present invention by a user.

FIG. 4 depicts a screenshot of the mobile device application of the system of the present invention, showing a medication and dosage screen.

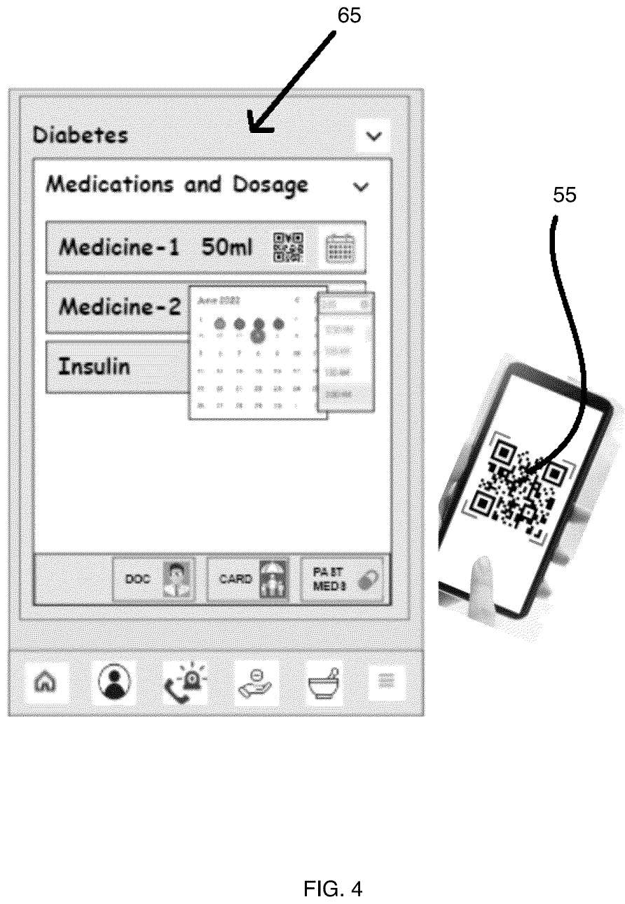

FIG. 5 depicts a close-up view of the base of the medicine container of the preferred embodiment of the present invention as seen with the container removed from the base, showing a top-down cutaway view of the base.

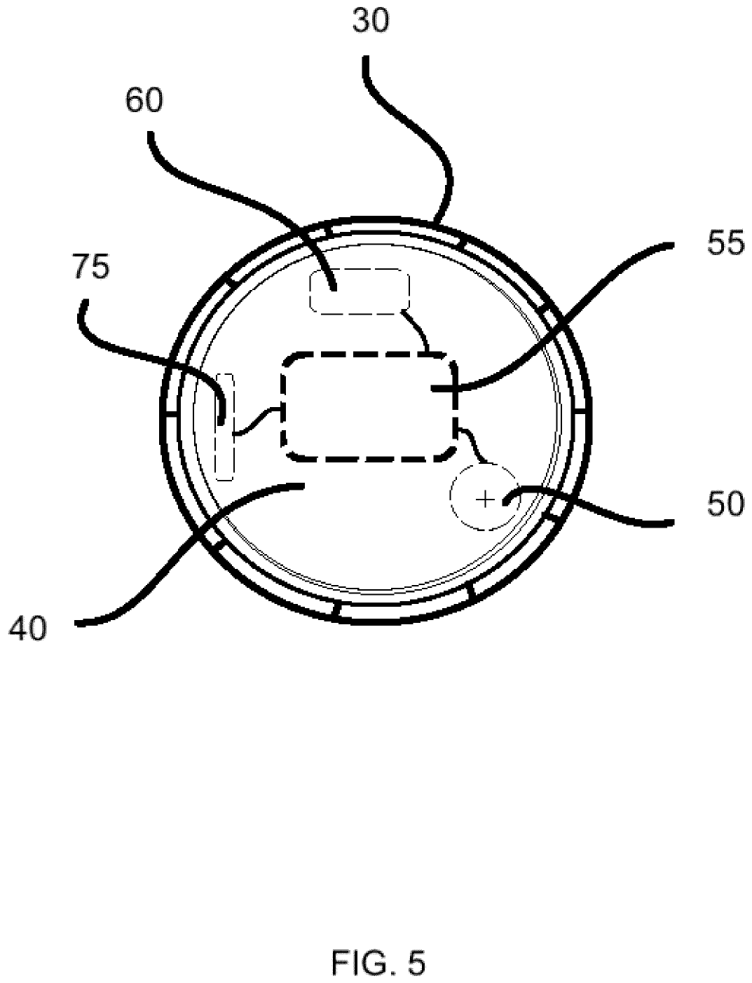

FIG. 6 depicts a modified blister-pack container equipped with the medication intake tracking system of the present invention.

### DETAILED DESCRIPTION OF THE PRESENT INVENTION

The present specification discloses one or more embodiments that incorporate the features of the invention. The disclosed embodiment(s) merely exemplify the invention. The scope of the invention is not limited to the disclosed embodiment(s).

References in the specification to “one embodiment,” “an embodiment,” “an example embodiment,” etc., indicate that the embodiment described may include a particular feature, structure, or characteristic, but every embodiment may not necessarily include the particular feature, structure or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The present invention is a system and apparatus configured to facilitate the tracking of medication intake by a patient. The apparatus of the present invention is integrated into a container (10) having a lid (20) and a base (30). The base (30) of the container (10) preferably houses the apparatus of the present invention entirely, however in some alternate embodiments, the lid (20) predominantly houses the apparatus of the present invention. The base (30) is preferably equipped with a scale (40), battery (50), motion detector (75) (and/or light sensor) and a Bluetooth™ radio (60). The scale (40) is a digital scale which is disposed in communication with the battery (50). The Bluetooth radio (60) is also disposed in communication with the battery (50). As the scale (40) is preferably disposed within the lid (20), the patient must invert the container (10) in order to obtain an accurate weight reading. Alternately, in some embodiments, the scale (40) is disposed at the lid (20) of the container (10). It should be understood that each embodiment of the container (10) contains medicine (25).

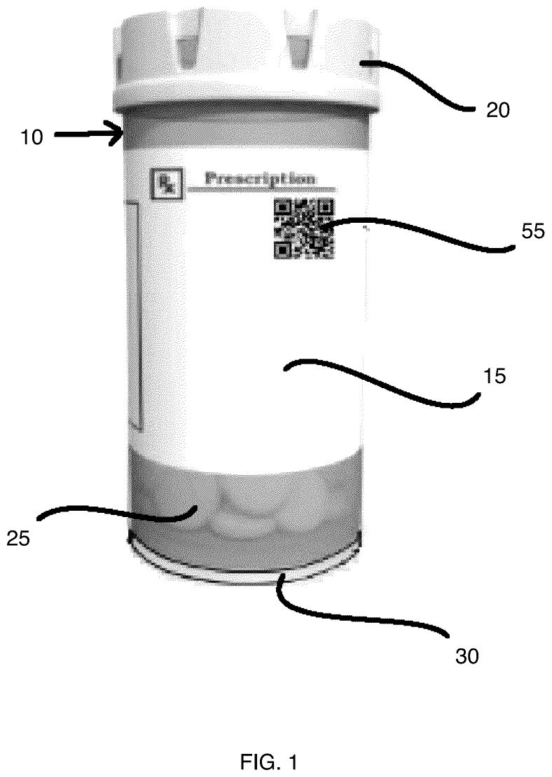

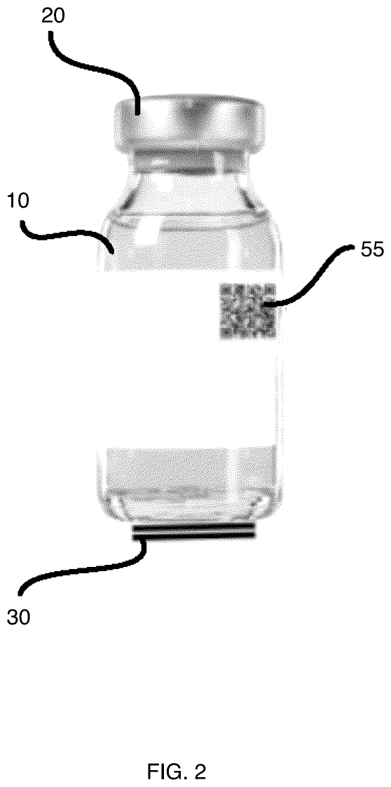

As shown in FIG. 1 and FIG. 2, the apparatus of the present invention is preferably integrated into a lid or a base of a conventional medicine bottle or vial. As such, the present invention is configured to function the same for liquid medicines (such as cough syrups, IV medications, etc.) as for those disposed in pill form conventionally disposed in medicine containers or blister packs. The container (10) is preferably equipped with a label (15) which exhibits a unique QR code (55) which is configured to facilitate the linking of the specific medication, including the prescribed dosage and frequency of intake information, to a mobile device application (65) to log the medication prescription data and medication intake data in real time.

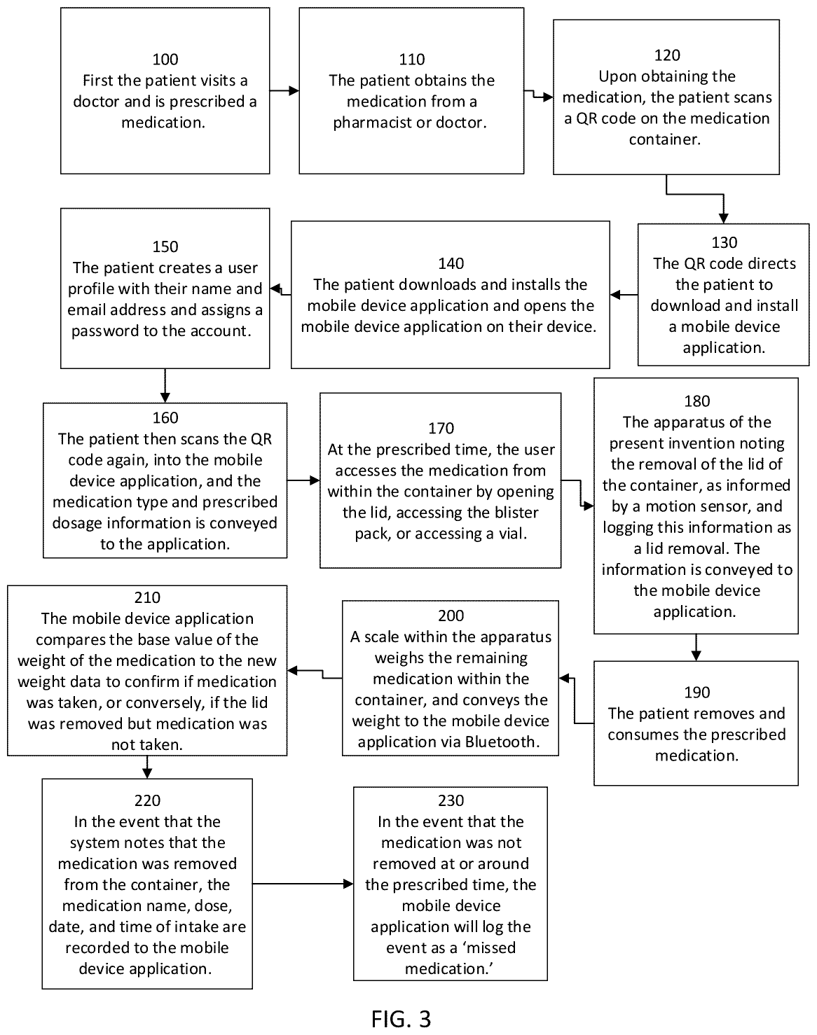

As outlined in FIG. 3, the process of use of the apparatus of the present invention by a user is preferably as follows:
1. First the patient visits a doctor and is prescribed a medication. (100)
2. The patient obtains the medication from a pharmacist or doctor. (110) The medication is disposed in a container (10) equipped with the apparatus of the present invention.
3. Upon obtaining the medication, the patient scans a QR code on the medication container. (120)
4. The QR code directs the patient to download and install a mobile device application. (130) (If the application is not detected as already installed to the device.)
5. The patient downloads and installs the mobile device application and opens the mobile device application on their device. (140) In the event that the patient already has the application installed to their mobile device, the act of scanning the QR code will automatically import prescription data to the mobile device.
6. The patient creates a user profile with their name and email address and assigns a password to the account. (150) In the event that the patient already has the application installed and an account previously created, this step is not necessary.
7. The patient then scans the QR code again, into the mobile device application, and the medication type and prescribed dosage information is conveyed to the application. (160) Setup is now complete. The initial weight of the medicine within the container is logged as a base value. Additional prescribed medication can be subsequently added by repeating step 7. To reiterate, in the event that the patient already has the application installed to their mobile device, the act of scanning the QR code the first time will automatically import prescription data to the mobile device, eliminating the need to scan the QR code a second time.
8. At the prescribed time, the user accesses the medication from within the container by opening the lid, accessing the blister pack, or accessing a vial. (170). The mobile device application may be configured to remind the patient at prescribed dosage times, or patient-selected dosage times if desired.
9. The apparatus of the present invention noting the removal of the lid of the container, as informed by a motion sensor and/or light sensor, and logging this information as a lid removal. The information is conveyed to the mobile device application. (180) The time of lid removal is logged in a data set.
10. The patient removes and consumes the prescribed medication. (190)
11. A scale within the apparatus weighs the remaining medication within the container, and conveys the current weight to the mobile device application via Bluetooth. (200)
12. The mobile device application compares the base value of the weight of the medication to the current weight value data to confirm if medication was taken, or conversely, if the lid was removed but medication was not taken. (210) The current weight data is then used as the new base value of the weight of the medication.
13. In the event that the system notes that the medication was removed from the container, the medication name, dose, date, and time of intake are recorded to the mobile device application. (220) The dose taken is inferred via the difference in weight between the base weight value and the current weight value.
14. In the event that the medication was not removed at or around the prescribed time, the mobile device application will log the event as a ‘missed medication.’ (230)

It should be noted that alternate embodiments of the present invention may exhibit different colors, textures, and patterns depicted on the container (10). Further, alternate embodiments of the present invention may exhibit a larger or smaller container (10) than shown in FIG. 1 and FIG. 2. Further, it should be understood that all embodiments of the apparatus of the present invention need not be equipped with a motion sensor disposed in the lid (20) of the container (10), but alternately, a light sensor may be present in the base (30) of the container. In the event that the container (10) is out of range of the mobile device of the user at the time of medication intake, the apparatus is configured to save the container access data (weight and/or motion detection data) to be conveyed later to the mobile device application via Bluetooth radio (60). A Programmable Logic Controller (PLC) (55) or similar logic controller is preferably present within the apparatus to facilitate and monitor connection between the scale (40), battery (50), and Bluetooth radio (60). The PLC may be equipped with onboard memory to ensure adequate temporary storage of medication access data.

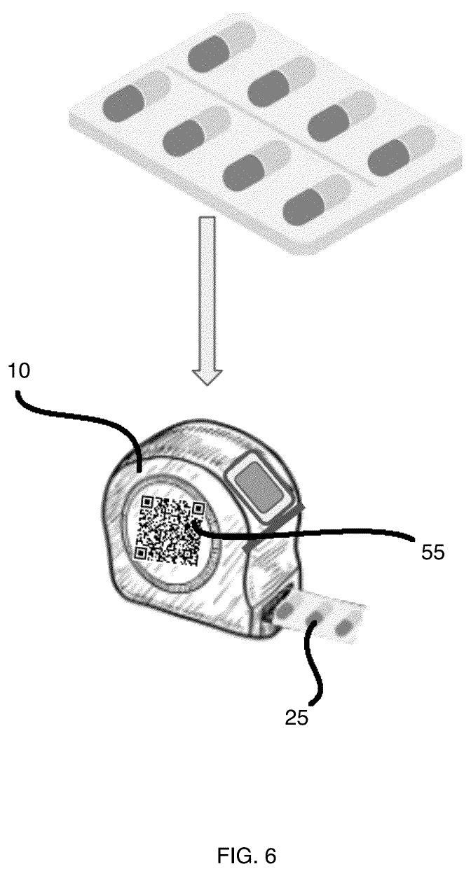

Further, it should be noted that in embodiments configured to facilitate the tracking of medications disposed in blister packs, the apparatus is preferably configured to manually count the remaining medications in real time. This is preferably assisted via a dispenser-type of packaging, similar in stature to that of a tape measure. In such embodiments, as shown in FIG. 6, no scale (40) is required. In embodiments configured to be integrated into a liquid medicine container (10), such as a vial or bottle, the scale (40) is preferably integrated into the base (30) of the container (10), rather than the lid (20).

Data collected by the system of the present invention is envisioned to assist in scientific research on multi-medication use for chronic conditions in patients. Additionally, if the patient permits and makes the intake data available to the physician and/or pharmaceutical company, the data may be used to monitor for any adverse events, assist in post-market clinical research for the medication, and further help the industry to predict drug shortages. Likewise, it should be noted that medication intake data can be seen by the patient from within the mobile device application (65) within a ‘calendar’ view, which indicates when medications were taken on which days, and when/if doses were missed. Further, a “Past Medications” button within the mobile device application (65) shows the patient any and all medications that the patient has been prescribed over the course of their lifetime (or as long as they have been using the system and platform of the present invention). In preferred embodiments, the mobile device application (65) preferably informs the patient of pertinent clinical trials in their area, and may encourage or inform the patient as to how one applies to take part in such trials. Similarly, any adverse effects experienced by the patient may be logged within the mobile device application (65), and alternately, the mobile device application (65) is configured to inform the patient of any known potential adverse effects (side effects) to the intake of the prescribed medication.

Having illustrated the present invention, it should be understood that various adjustments and versions might be implemented without venturing away from the essence of the present invention. Further, it should be understood that the present invention is not solely limited to the invention as described in the embodiments above, but further comprises any and all embodiments within the scope of this application.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

I claim:

1. A medication tracking system for implementation on a medicine container containing medicine, the system configured to interface with a mobile device comprising:
a medicine bottle lid, said medicine bottle lid equipped with a programmable logic controller, a digital scale, a battery, a motion detector, and a Bluetooth radio;
wherein said digital scale, said battery, said motion detector, and said Bluetooth radio are disposed in communication with said programmable logic controller;
a label, said label disposed on the medicine container;
a QR code, said QR code disposed on said label;
a mobile device application, said mobile device application installed to and running on the mobile device;
wherein said QR code, when scanned, imports data pertaining to the medication of the medicine container;
wherein said digital scale is configured to weigh the remaining medicine of the medicine container when the medicine container is inverted; and
wherein said programmable logic controller is configured to employ said Bluetooth radio to convey the weight of the remaining medicine to the mobile device and display the weight on the mobile device application.

2. A method for tracking doses of medication from a medicine container, the method comprising:
a patient being prescribed medication in the medicine container;
the medicine container being equipped with a medicine container lid;
wherein the medicine container lid is equipped with a programmable logic controller disposed in communication with a battery, a Bluetooth radio, a digital scale, and a light sensor;
the patient scanning a QR code on a label disposed on the medicine container;
the QR code directing the patient to download and install a mobile device application;
the patient downloading and installing the mobile device application to their mobile device;
the patient opening the mobile device application on their mobile device;
the patient scanning the QR code a second time within the mobile device application;
the mobile device application interpreting the data of the QR code, thereby importing a medication type and prescribed dosage information specific to the patient;
the digital scale logging the initial weight of the medicine within the medicine container as a base value;
at a prescribed time interval, the mobile device application reminding the patient to take the medicine of the medicine container;
the patient opening the lid of the medicine container;
the light sensor noting the change in light value upon the container opening, logging an opening of the container to the programmable logic controller;
the programmable logic controller conveying the opening of the container to the mobile device application via the Bluetooth radio;
the patient removing and consuming the prescribed medication;
the digital scale weighing the remaining medication within the medicine container;
the programmable logic controller logging the weight of the remaining medication;
the programmable logic controller relaying the weight of the remaining medication to the mobile device application via the Bluetooth radio;
the mobile device application comparing the base value of the weight of the medication to the updated weight of the medication to confirm if medication was taken;
the mobile device application comparing the base value of the weight of the medication to the updated weight of the medication to determine if medication was not taken;
the mobile device application comparing the base value of the weight of the medication to the updated weight of the medication to determine if more medication was taken than the prescribed dose;
the mobile device application recording that the medication was removed from the container, including the medication name, dose, date, and time of intake in the event that the weight of the remaining medication is lower than the base value of the weight of the medication;
the mobile device application recording that the medication was not removed from the container, including the medication name, dose, date, and time of failed intake in the event that the weight of the remaining medication is the same as the base value of the weight of the medication; and
the digital scale using the updated weight of the medication as a new base value weight of the medication.

3. A medication tracking system for implementation on a medicine container containing medicine, the system configured to interface with a mobile device comprising:
a medicine bottle base, said medicine bottle base equipped with a programmable logic controller, a digital scale, a battery, a motion detector, and a Bluetooth radio;
wherein said digital scale, said battery, said motion detector, and said Bluetooth radio are disposed in communication with said programmable logic controller;
a label, said label disposed on an exterior of the medicine container;
a QR code, said QR code disposed on said label;
a mobile device application, said mobile device application installed to and running on the mobile device;
wherein said QR code, when scanned, imports data pertaining to the medication of the medicine container;
wherein said digital scale is configured to weigh the remaining medicine of the medicine container; and
wherein said programmable logic controller is configured to employ said Bluetooth radio to convey the weight of the remaining medicine to the mobile device and display the weight on the mobile device application.

4. The medication tracking system of claim 3, wherein said mobile device application is configured to remind the patient of prescribed dosage times.

5. A medication tracking system for use by a patient comprising:
a container having a lid and a base, wherein said container is configured to house medication;
a scale integrated within said container to measure a weight of the medication, said scale being disposed in communication with a battery;
a light sensor configured to detect removal of said lid of said container;
a Bluetooth radio in communication with said battery and configured to transmit data;
a mobile device application configured to receive data from said Bluetooth radio, wherein said data includes weight measurements of the medication, and to log medication intake events based on the weight measurements and detected lid removal events; and
a unique QR code on a label of the container that, when scanned, facilitates linking the medication to the mobile device application for logging prescription and intake data in real time.

6. The system of claim 5, wherein said scale is integrated into said base of said container.

7. The system of claim 5, wherein said scale is integrated into said lid of said container.

8. The system of claim 5, wherein said light sensor is disposed in said base of the container.

9. The system of claim 5, wherein said light sensor is disposed in said lid of the container.

10. The system of claim 5, wherein said mobile device application is configured to remind the patient of prescribed dosage times.

11. The system of claim 5, further comprising: a programmable logic controller (PLC) within said container to facilitate and monitor the connection between the scale, battery, and Bluetooth radio.

12. The system of claim 5, wherein said Bluetooth radio is configured to store container access data when out of range of said mobile device and transmit the data to said mobile device application when within range.

13. The system of claim 5, wherein said mobile device application displays a calendar view indicating medication intake and missed doses.

14. The system of claim 5, wherein said mobile device application logs adverse effects experienced by the patient and informs the patient of known potential adverse effects associated with the medication.

15. The system of claim 6, further comprising: a programmable logic controller (PLC) within said container to facilitate and monitor the connection between the scale, battery, and Bluetooth radio.

16. The system of claim 15, wherein said light sensor is disposed in said base of the container.

17. The system of claim 16, wherein said mobile device application is configured to remind the patient of prescribed dosage times.

18. The system of claim 17, wherein said Bluetooth radio is configured to store container access data when out of range of said mobile device and transmit the data to said mobile device application when within range.

19. The system of claim 18, wherein said mobile device application displays a calendar view indicating medication intake and missed doses.

20. The system of claim 19, wherein said mobile device application logs adverse effects experienced by the patient and informs the patient of known potential adverse effects associated with the medication.
